(19) United States
(12) Patent Application Publication
(10) Pub. No.: US 2025/0259420 A1
(43) Pub. Date: Aug. 14, 2025

(54) SYSTEMS AND METHODS FOR METADATA-BASED ANATOMY RECOGNITION

(71) Applicant: Blackford Analysis Ltd., Edinburgh (GB)

(72) Inventors: Robert John TWEEDIE, Edinburgh (GB); Conrad Blair CHIN, Edinburgh (GB); Andrew MURRAY, Edinburgh (GB); James CADMAN, London (GB)

(21) Appl. No.: 19/061,121

(22) Filed: Feb. 24, 2025

Related U.S. Application Data
(63) Continuation of application No. PCT/EP2023/072344, filed on Aug. 11, 2023.
(60) Provisional application No. 63/399,955, filed on Aug. 22, 2022.

(51) Int. Cl. G06V 10/764; G16H 30/20; G16H 30/40

(52) U.S. Cl. CPC G06V 10/764; G16H 30/20; G16H 30/40; G06V 2201/03

(57) ABSTRACT

Provided are computer-implemented systems and methods for metadata-based anatomy recognition and computer-implemented systems and methods for generating a model for metadata-based anatomy recognition. The metadata-based anatomy recognition includes: providing, in a memory in communication with a processor, a model for metadata-based anatomy recognition; receiving, using a network device in communication with the processor, at least one medical image object comprising a plurality of metadata; determining, at the processor, a predicted anatomy classification associated with the at least one medical image object based on the model for metadata-based anatomy recognition and the plurality of metadata; and storing, in the memory, the predicted anatomy classification in association with the at least one medical image object in a database.

### CROSS-REFERENCE TO RELATED APPLICATION

This application is a continuation of International Application No. PCT/EP2023/072344, filed Aug. 11, 2023, which claims the benefit of U.S. Provisional Patent Application No. 63/399,955, filed Aug. 22, 2022, the entire contents of each of which are incorporated by reference herein.

### FIELD

The described embodiments relate generally to the processing of medical image data using a plurality of clinical software applications, and specifically to the prediction of the anatomy of a medical image for processing using the plurality of clinical software applications.

### BACKGROUND

Large medical organizations have challenges in the routing of medical data between different systems involved in collecting the medical data, and clinical software applications that process and analyze the medical data. This is particularly challenging due to the fact that medical images may be very large. Furthermore, the medical organization may have many different information systems implemented to process data.

Medical data is collected about a patient when they are imaged at an image acquisition device, and the medical data includes image data and associated image metadata. “A modality” refers to the categorical type of image acquisition generated by different image acquisition devices (e.g. CT, MR, x-ray, ultrasound or PET; as in, “those images have modality ‘CT’”). Depending on diagnostic or clinical need and modality type, a scanning episode might capture a single image, a series of images covering one area of anatomy, or multiple series of images covering the same or different areas of anatomy, the latter for example if there are multiple modes of operation for a scanner (e.g. MR) or perhaps to cover the time before and after administration of a contrast agent. Furthermore, multiple scans may be taken of the same patient and anatomy area at different times, using the same or different image acquisition device. Finally, some image acquisition devices allow for the capture of videos of moving anatomy (e.g. a beating heart)—which herein is treated as a series of images amenable to processing.

A single patient may be imaged by an image acquisition device. The patient may visit and be imaged by the image acquisition device on multiple different occasions, and these studies may be for a planned sequence of scans to monitor a disease and/or to track treatment. This may be referred to as a longitudinal sequence of studies. At the medical provider, IT systems may be provided to store the studies and associate individual studies of the same patient with one another. However, between different IT systems (or even between different studies on the same IT system) patient medical data may not be correctly associated. It is often desirable to perform additional analysis on images after acquisition at the image acquisition device, but before presentation to clinicians. Such processing analyses have the aim of supporting a clinician's review of the scan by, for example, improving the speed of a clinician's review. This may further include identifying certain clinically relevant features in the images, or adding new, clinically useful, images derived in some way from the originals.

A first challenge in the medical organization is how to process collected studies that include different anatomical views using a plurality of clinical applications. There may be many clinical applications that may add, adjust, edit, merge, analyze, or otherwise process image and metadata collected in studies. The clinical applications in such a system may update the study record, including the image data and metadata, or may create new images or studies entirely. The anatomical view of the study is important for particular analyses or diagnostic tasks. For example, a clinical application may be particularly designed to identify and flag cancer in the head or neck of a subject. In order to determine the data to use for processing with such a clinical application, it is important to know that the relevant studies or images being processed are of the right anatomical view to support the analysis. During processing using a clinical software application, it is desirable to have a complete set of all matching studies and images. Existing solutions do not adequately address the challenges faced when trying to collect medical data for processing by clinical software applications that include particularly relevant anatomical views.

In medical organizations, the metadata identifying anatomical view is often input by hand. It is often of a non-standard format which may vary from one medical imaging device manufacturer to another and also varying by institution. The net result is that existing study metadata is unreliable since it may be missing or erroneous.

Prior systems for identifying the anatomy of a particular study require the processing of pixel data associated with the study, which adds significant processing overhead (both in terms of network traffic and in terms of the computational complexity of automatically identifying an anatomical view of the study from the pixel data). Such a conventional pixel-based system may include an Unsupervised Body Part Regressor.

Prior solutions allowed for workflow routing, where studies including medical data are sent from inside the medical organization to an identified clinician (or group of clinicians) at the time they need it. Such prior solutions, however, are not concerned with the problem of getting the right medical image data (based on anatomical view) to a clinical software application that performs analyses related to the anatomical view based on the images and produces a processing result.

Unlike clinician users, the clinical software applications do not move around physically, nor are they generally able to identify the anatomical view of studies or images. Clinician users generally select relevant or related studies to receive next from a user interface available to the clinician, and make their own decisions which data to transfer for review. Clinical software applications may have significant computer resource requirements, and these resource requirements may have costs associated with their use, for example, the use of a clinical software application running on a cloud provider such as Amazon® Web Services (AWS®). There is a need therefore to avoid processing using clinical applications based on irrelevant studies, series, or image data which contain anatomical views unrelated to the processing requirements of a clinical application.

A second challenge in the medical organization is improving clinician workflows. This can include the need to avoid unnecessary pauses by the clinician during the speed of a clinician's review by ensuring that particular studies for a subject are routed to the clinician's workstation appropriately for the purposes of each particular review. For example, for a clinician who is tasked with reviewing a subject's studies for a brain tumor, the clinician would need to have neck and head studies or images routed to their workstation to avoid unnecessary pauses by the clinician during network file transfer. The identification of the anatomical view of particular studies is thus required to reduce the file transfer overhead required for a clinician to review studies for a subject.

Medical images may be very large in size, and the transfer of the medical images over a network very costly. There is a need therefore to ensure that relevant and related studies, series, and images of particular anatomical views are transferred efficiently to avoid unnecessary resource usage.

A third challenge, related to the second challenge, is improving clinician workflows by automating the display of medical images at a clinician's workstations. Clinicians frequently review medical studies and images using a hanging protocol. A hanging protocol is the series of actions performed to arrange images for optimal softcopy viewing. The term “hanging protocol” originally referred to the arrangement of physical films on a light box or hanging of films on a film alternator. Now the term may refer to displaying softcopy images on a display device of a clinician workstation. The goal of a hanging protocol is to present specific types of studies in a consistent manner, and to reduce the number of manual image ordering adjustments performed by the clinician.

Hanging protocols may vary based on modality, anatomical part, department, personal preference, and even training. For example, one clinician may want to look at a chest image series with the posteroanterior (PA) view on the left, while another radiologist prefers the PA view on the right.

On a clinician workstation, an appropriate hanging protocol may be automatically applied based on the characteristics of the study being loaded. For the appropriate hanging protocol to be automatically applied however, information such as modality, anatomical part, study or series description must be available to ensure proper selection. Such information however is not always available.

In addition, information such as series IDs, image orientation (image laterality and view code), and patient positioning may be used to organize the images properly, if such information is available.

The DICOM standard includes a Hanging Protocol Service Class and a Hanging Protocol Composite IOD.

There is a need therefore to ensure that anatomical information for each study or image is correctly applied, so that a hanging protocol can be automatically applied at a clinician workstation such that the clinician can efficiently review the studies or images together.

### SUMMARY

Fetching pixel data to infer the anatomy of a medical study or medical image is expensive (in terms of elapsed time, processing effort and burden on existing systems). Further, fetching pixel data just to infer anatomy might mean that lots of pixel data is requested and received that is not actually needed for processing.

Metadata-based anatomy inferences can address these challenges. To infer anatomy from the metadata alone, an existing anatomy model that uses pixel data may be used as a ground truth for the generation of a metadata-based model.

In a first aspect, there is provided a computer-implemented method for metadata-based anatomy recognition, comprising: providing, in a memory in communication with a processor, a model for metadata-based anatomy recognition; receiving, using a network device in communication with the processor, at least one medical image object comprising a plurality of metadata; determining, at the processor, a predicted anatomy classification associated with the at least one medical image object based on the model for metadata-based anatomy recognition and the plurality of metadata; and storing, in the memory, the predicted anatomy classification in association with the at least one medical image object in a database.

In one or more embodiments, the model for metadata-based anatomy recognition may comprise a model for tag-based anatomy recognition; the plurality of metadata may comprise a plurality of tag-based metadata; and the predicted anatomy classification may be determined based on the model for tag-based anatomy recognition and the plurality of tag-based metadata.

In one or more embodiments, the model for tag-based anatomy recognition may comprise a Digital Imaging and Communications in Medicine (DICOM)-based model for tag-based anatomy recognition; the plurality of tag-based metadata may comprise a plurality of DICOM metadata; and the predicted anatomy classification may be determined based on the DICOM-based model for tag-based anatomy recognition and the plurality of DICOM metadata.

In one or more embodiments, the method may further include: generating a matched study set comprising the at least one medical image object and the predicted anatomy classification; determining a first clinical application based on the at least one medical image object and the model for metadata-based anatomy recognition; and transmitting the matched study set to the first clinical application.

In one or more embodiments, the method may further include: displaying, at a display device in communication with the processor, pixel data corresponding to the at least one medical image object; wherein the predicted anatomy classification may determine the display of the pixel data on the display device.

In one or more embodiments, the at least one medical image object may be received from a Picture Archiving and Communication Systems (PACS) server or a medical imaging device.

In one or more embodiments, the method may further include: determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network; comparing the predicted anatomy classification to the pixel-based predicted anatomy classification; and if the pixel-based predicted anatomy classification is different from the pixel-based predicted anatomy classification, may include flagging the at least one medical image object for review.

In one or more embodiments, the method may further include: determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network; comparing the predicted anatomy classification to the pixel-based predicted anatomy classification; and if the predicted anatomy classification is different from the pixel-based predicted anatomy classification, may automatically retrain the model for metadata-based anatomy recognition based on the pixel-based predicted anatomy classification and the at least one medical image object.

In one or more embodiments, the method may further include: determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network; comparing the predicted anatomy classification to the pixel-based predicted anatomy classification; and if the predicted anatomy classification is different from the pixel-based predicted anatomy classification, may automatically retrain the model for pixel-based anatomy recognition based on the predicted anatomy classification and the at least one medical image object.

In one or more embodiments, the method may further include: determining, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network; comparing the second predicted clinical application to the first predicted clinical application; and if the first predicted clinical application is different from the second predicted clinical application, may flag the at least one medical image object for review.

In one or more embodiments, the method may further include: determining, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network; comparing the second predicted clinical application to the first predicted clinical application; and if the first predicted clinical application is different from the second predicted clinical application, may automatically retrain the model for metadata-based anatomy recognition based on the pixel-based predicted anatomy classification and the at least one medical image object.

In one or more embodiments, the method may further include: determining, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network; comparing the second predicted clinical application to the first predicted clinical application; and if the first predicted clinical application is different from the second predicted clinical application, may automatically retrain the model for pixel-based anatomy recognition based on the predicted anatomy classification and the at least one medical image object.

In one or more embodiments, the model for metadata-based anatomy recognition may comprise a Recurrent Neural Network (RNN) model, and optionally a Long Short Term Memory (LSTM) model, a random forest model, a decision tree model, or a fully connected network model.

In a second aspect, there is provided a computer-implemented system for metadata-based anatomy recognition, comprising: a memory, comprising: a model for metadata-based anatomy recognition; a network device, and a processor, the processor configured to: receive, from the network device, at least one medical image object comprising a plurality of metadata; determine a predicted anatomy classification associated with the at least one medical image object based on the model for metadata-based anatomy recognition and the plurality of metadata; and store, in the memory, the predicted anatomy classification in association with the at least one medical image object in a database.

In one or more embodiments, the model for metadata-based anatomy recognition may comprise a model for tag-based anatomy recognition; the plurality of metadata may comprise a plurality of tag-based metadata; and the predicted anatomy classification may be determined based on the model for tag-based anatomy recognition and the plurality of tag-based metadata.

In one or more embodiments, the model for tag-based anatomy recognition may comprise a Digital Imaging and Communications in Medicine (DICOM)-based model for tag-based anatomy recognition; the plurality of tag-based metadata may comprise a plurality of DICOM metadata; and the predicted anatomy classification may be determined based on the DICOM-based model for tag-based anatomy recognition and the plurality of DICOM metadata.

In one or more embodiments, the processor may be further configured to: generate a matched study set comprising the at least one medical image object and the predicted anatomy classification; determine a first clinical application based on the at least one medical image object and the model for metadata-based anatomy recognition; and transmit the matched study set to the first clinical application.

In one or more embodiments, the system may further comprise: a display device in communication with the processor; wherein the processor may be further configured to: display, at the display device, pixel data corresponding to the at least one medical image object; and wherein the predicted anatomy classification may determine the display of the pixel data on the display device.

In one or more embodiments, the at least one medical image object may be received from a Picture Archiving and Communication Systems (PACS) server or a medical imaging device.

In one or more embodiments, the processor may be further configured to: determine a pixel-based predicted anatomy classification from a model for pixel-based anatomy recognition, wherein the model for pixel-based anatomy recognition optionally comprises an Unsupervised Body-part Regressor (UBR) or a Convolutional Neural Network; compare the predicted anatomy classification to the pixel-based predicted anatomy classification; and if the pixel-based predicted anatomy classification is different from the pixel-based predicted anatomy classification, may flag the at least one medical image object for review.

In one or more embodiments, the processor may be further configured to: determine a pixel-based predicted anatomy classification from a model for pixel-based anatomy recognition, wherein the model for pixel-based anatomy recognition optionally comprises an Unsupervised Body-part Regressor (UBR) or a Convolutional Neural Network; compare the predicted anatomy classification to the pixel-based predicted anatomy classification; and if the predicted anatomy classification is different from the pixel-based predicted anatomy classification, may automatically retrain the model for metadata-based anatomy recognition based on the pixel-based predicted anatomy classification and the at least one medical image object.

In one or more embodiments, the processor may be further configured to: determine a pixel-based predicted anatomy classification from a model for pixel-based anatomy recognition, wherein the model for pixel-based anatomy recognition optionally comprises an Unsupervised Body-part Regressor (UBR) or a Convolutional Neural

Network; compare the predicted anatomy classification to the pixel-based predicted anatomy classification; and if the predicted anatomy classification is different from the pixel-based predicted anatomy classification, may automatically retrain the model for pixel-based anatomy recognition based on the predicted anatomy classification and the at least one medical image object.

In one or more embodiments, the processor may be further configured to: determine a pixel-based predicted anatomy classification and a second predicted clinical application from a model for pixel-based anatomy recognition. wherein the model for pixel-based anatomy recognition optionally comprises an Unsupervised Body-part Regressor (UBR) or a Convolutional Neural Network; compare the second predicted clinical application to the first predicted clinical application; and if the first predicted clinical application is different from the second predicted clinical application, may flag the at least one medical image object for review.

In one or more embodiments, the processor may be further configured to: determine a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network; compare the second predicted clinical application to the first predicted clinical application; and if the first predicted clinical application is different from the second predicted clinical application, may automatically retrain the model for metadata-based anatomy recognition based on the pixel-based predicted anatomy classification and the at least one medical image object.

In one or more embodiments, the processor may be further configured to: determine, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network; compare the second predicted clinical application to the first predicted clinical application; and if the first predicted clinical application is different from the second predicted clinical application, may automatically retrain the model for pixel-based anatomy recognition based on the predicted anatomy classification and the at least one medical image object.

In one or more embodiments, the model for metadata-based anatomy recognition may comprise a Recurrent Neural Network (RNN) model and optionally a Long Short Term Memory (LSTM) model, a random forest model, a decision tree model, or a fully connected network model.

In a third aspect, there is provided a computer-implemented method for generating a model for metadata-based anatomy recognition, comprising: providing, in a memory in communication with a processor, at least one medical image object comprising pixel data and a plurality of metadata; determining, at the processor, at least one anatomy classification corresponding to the at least one medical image object based on the corresponding pixel data and a pixel-based anatomy model; generating, at the processor, a model for metadata-based anatomy recognition based on the at least one anatomy classification and the plurality of metadata, the model for metadata-based anatomy recognition providing metadata-based anatomy predictions; and storing, in the memory, the model for metadata-based anatomy recognition.

In one or more embodiments, the method may further include: receiving the at least one medical image object from a PACS server or a medical imaging device using a network device in communication with the processor.

In one or more embodiments, the model for metadata-based anatomy recognition may comprise a model for tag-based anatomy recognition; the plurality of metadata may comprise a plurality of tag-based metadata; and the predicted anatomy classification may be determined based on the model for tag-based anatomy recognition and the plurality of tag-based metadata.

In one or more embodiments, the model for tag-based anatomy recognition may comprise a model for Digital Imaging and Communications in Medicine (DICOM)-based anatomy recognition; the plurality of tag-based metadata may comprise a plurality of DICOM metadata; and the predicted anatomy classification may be determined based on the model for DICOM-based anatomy recognition and the plurality of DICOM metadata.

In one or more embodiments, the pixel-based anatomy model may comprise an Unsupervised Body-part Regressor (UBR) or a Convolutional Neural Network.

In one or more embodiments, the model for metadata-based anatomy recognition may comprise a Recurrent Neural Network (RNN) model and optionally a Long Short Term Memory (LSTM) model, a random forest model, a decision tree model, or a fully connected network model.

In a fourth aspect, there is provided a computer-implemented system for generating a model for metadata-based anatomy recognition, comprising: a memory comprising: at least one medical image object comprising: pixel data, and a plurality of metadata; a network device, and a processor configured to: determine at least one anatomy classification corresponding to the at least one medical image object based on the corresponding pixel data and a pixel-based anatomy model; generate a model for metadata-based anatomy recognition based on the at least one anatomy classification and the plurality of metadata, the model for metadata-based anatomy recognition providing metadata-based anatomy predictions; and store, in the memory, the model for metadata-based anatomy recognition.

In one or more embodiments, the processor may be further configured to: receive the at least one medical image object from a PACS server or a medical imaging device using the network device.

In one or more embodiments, the model for metadata-based anatomy recognition may comprise a model for tag-based anatomy recognition; the plurality of metadata may comprise a plurality of tag-based metadata; and the predicted anatomy classification may be determined based on the model for tag-based anatomy recognition and the plurality of tag-based metadata.

In one or more embodiments, the model for tag-based anatomy recognition may comprise a model for Digital Imaging and Communications in Medicine (DICOM)-based anatomy recognition; the plurality of tag-based metadata may comprise a plurality of DICOM metadata; and the predicted anatomy classification may be determined based on the model for DICOM-based anatomy recognition and the plurality of DICOM metadata.

In one or more embodiments, the pixel-based anatomy model may comprise an Unsupervised Body-part Regressor (UBR) or a Convolutional Neural Network.

In one or more embodiments, the model for metadata-based anatomy recognition may comprise a Recurrent Neural Network (RNN) model and optionally a Long Short Term Memory (LSTM) model, a random forest model, a decision tree model, or a fully connected network model.

### BRIEF DESCRIPTION OF THE DRAWINGS

A preferred embodiment of the present invention will now be described in detail with reference to the drawings, in which:

FIG. 1 is a system diagram in accordance with one or more embodiments;

FIG. 2 is a server diagram in accordance with one or more embodiments;

FIG. 3 is a block diagram of a metadata object model in accordance with one or more embodiments;

FIG. 4A is an example of medical image data in accordance with one or more embodiments;

FIG. 4B is another example of medical image data in accordance with one or more embodiments;

FIG. 5A is a method diagram for anatomy recognition in accordance with one or more embodiments;

FIG. 5B is a predicted anatomy diagram for an Unsupervised Body Part Regressor in accordance with one or more embodiments;

FIG. 6 is a method diagram in accordance with one or more embodiments;

FIG. 7 is another method diagram in accordance with one or more embodiments; and

FIG. 8 is a user interface diagram in accordance with one or more embodiments.

### DESCRIPTION OF EXEMPLARY EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural, declarative, functional or object-oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims. Also, in the various user interfaces illustrated in the figures, it will be understood that the illustrated user interface text and controls are provided as examples only and are not meant to be limiting. Other suitable user interface elements may be possible.

As described herein, “DICOM” refers to the Digital Imaging and Communications in Medicine (DICOM) standard for the communication and management of medical imaging information and related data as published by the National Electrical Manufacturers Association (NEMA).

As described herein, “HL7” refers to the Health Level 7 (HL7) standard as published by Health Level Seven International.

As described herein, “medical images”, “image data”, or “images” refers to image data collected by image acquisition devices, also known as “instances”. The images are visual representations of the interior of a body anatomy that may be used for clinical analysis and medical interventions, commonly referred to as radiology. Radiology may use the imaging technologies including X-ray Plain Film (PF), digital X-rays, Cardiology imaging devices including a cardiology PACS server, Computed Tomography (CT) images, ultrasound images, nuclear medicine imaging including Positron-Emission Tomography (PET), Veterinary imaging devices, Magnetic Resonance Imaging (MRI) images, mammographic images, or any other standardized images used in a medical organization. Medical imaging also establishes a database of normal anatomy and physiology to make it possible to identify abnormalities. Medical imaging may further include removed organs and tissues. The medical images may be collected using analog means such as film and then subsequently scanned, or may be constructed from data originally collected using digital sensor means such as a Charge Coupled Device (CCD) and processed into image data. The image data may be provided in JPEG, JFIF, JPEG2000, Exif, GIF, BMP, PNG, PPM, PGM, PBM, PNM, WebP, HDR, HEIF, or any other known format. The image data may be provided in an uncompressed image format, in a lossless compressed format, or in a lossy compressed format.

While existing data processing systems generally provide non-contextual processing, and do not determine the relevancy of one study to another for processing, the processing of the clinical software applications herein includes information enhancement that are only relevant to specific types of data, perform more efficiently with specific, relevant data, and indeed the processing may not function properly at all without such specific relevant data.

The clinical software applications of the medical image processing system may provide clinically relevant information, such as by adding new medical images and image metadata. This information enhancement may include adding metadata fields, manipulating images, and associating a plurality of studies, series, and image data and metadata together from disparate sources.

As described herein, clinical software applications are software applications that generate additional information about a medical study or studies through the analysis of metadata and/or the image data including pixel data of one or more images of the study or studies. Such analysis might range from simple to highly complex calculations including statistical inferences and machine learning. The analysis by the clinical software application may be “global” in the sense that it takes account of pixels and/or meta-data across an entire matched set of images, or several sets of images. The clinical software application analysis may add clinically relevant information as described herein. Alternatively, the analysis may be a “local” analysis that, e.g. only performs determinations based on a single image or fragment of meta-data. Further, the clinical software application may add information, including probabilistic information such as confidence levels, probabilities, and/or certainties, in addition to just performing a transformation of the data (for example, the transformation may go beyond simple image compression). The decision making and rule application for each clinical software application can be used for making “routing” decisions for determining the relevancy of a set of images to a particular clinical software application.

Specific examples of clinical software applications may include (but are not limited to): image registration, anatomy recognition, contrast detection, image quality, quantification of brain abnormalities and changes from MR images in order to track disease evolution, liver iron concentration analysis from MR images of the liver, and the removal of personally identifying information.

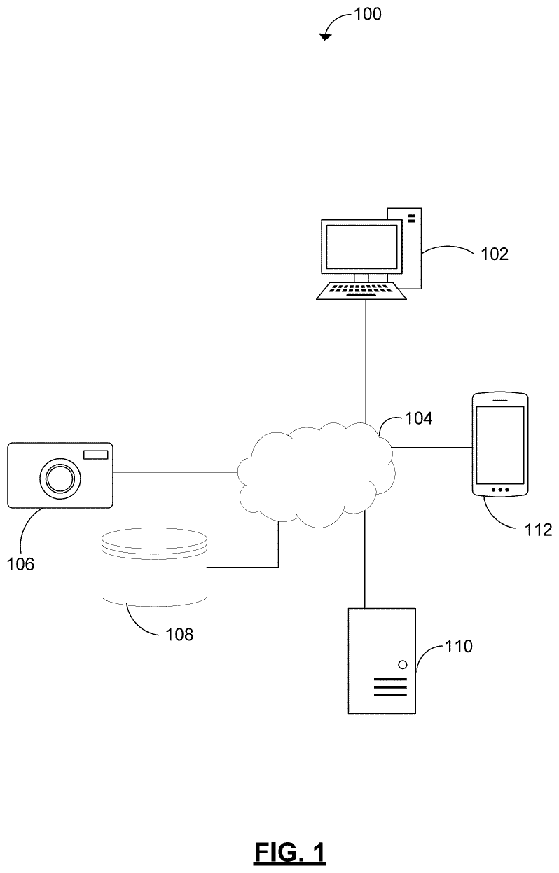

Reference is first made to FIG. 1, which illustrates a medical data processing system 100. The system 100 has a plurality of user devices including mobile device 112 and computer device 102, network 104, a server 110, an image acquisition device 106, and an enterprise imaging server 108. The system 100 may describe a medical organization that may include a network 104 that is inside the medical organization that provides interconnection. The medical data processing system may be at a medical organization, which may include one or more related medical organizations that share medical image data and associated metadata over one or more networks. The one or more related medical organizations may include one or more image acquisition devices, one or more geographical locations, a plurality of clinician users, a plurality of administrative users, and a plurality of patients attending the one or more image acquisition devices for medical imaging services.

In an alternate embodiment, the processing server 110 may be external to the medical organization and the imaging acquisition device 106 and enterprise imaging server 108 may forward image data and associated metadata to the server via network 104 and a firewall.

User devices including mobile device 112 and computer device 102 may be used by end users to access an application (not shown) running on server 110 over network 104. For example, the application may be a web application, or a client/server application. The user devices may display the application, and may allow a user to review medical data, including medical images and image metadata. The users at the user devices may be a clinician user at a medical organization who may review the medical data, including processed medical data from the clinical software applications. A clinician user may be a radiologist whose role is the review (or reading) of medical images, or a referring clinician (for example, the non-radiologist clinician who referred the patient for a scan) who may receive a report from the radiologist.

The users at user devices may be an administrator user who may administer the configuration of clinical software applications for the medical organization.

The enterprise imaging server 108 may be a Picture Archiving and Communication System (PACS) server, a Modality Worklist (MWL), or another medical image data archive. For example, an enterprise imaging device may be an IntelePACS® from Intelerad®, an IntelliSpace® PACS from Philips®, an enterprise imaging device such as the Enterprise Imaging Solution@ suite from Change Healthcare®. For example, an enterprise imaging device may be a Medicor® MiPACS® Modality Worklist. For example, an enterprise imaging device may be an IBM iConnect Enterprise Archive. An enterprise imaging server 108 may be remote from the medical organization. For example, a remote PACS may be at an affiliated medical organization to the medical organization, for example, a satellite clinic.

An enterprise imaging server 108 may provide economical storage and convenient access to medical images and image metadata from multiple image acquisition devices external to medical organization. A PACS may support live Query/Retrieve, archive Query/Retrieve, be configured to auto-forward, or a combination of these roles.

Enterprise imaging server 108 may be a Modality Worklist (MWL), where the MWL makes patient demographic information from a Radiology Information System (RIS) available at an image acquisition device, and providing, amongst other things, a worklist of patients who will attend the image acquisition device for imaging in the near future. The MWL may further provide in-progress studies and completed studies.

The enterprise imaging server 108 may store image metadata in a DICOM format, an HL7 format, an XML-based format, or any other format for exchanging image data and associated metadata.

Server 110 may be a commercial off-the-shelf server. In an alternate embodiment, the remote server 132 may be a server running on Amazon@ Web Services (AWS®) or another similar hosting service. The remote server 110 may be a physical server or may be a virtual server running on a shared host. The server 110 may have an application server, a web server, a database server, or a combination thereof. The application server may be one such as Apache Tomcat, etc. as is known. The web server may be a web server for static web assets, such as Apache® HTTP Server, etc. as is known. The database server may store user information including structured data sets, electronic form mappings, and other electronic form information. The database server may be a Structured Query Language (SQL) such as PostgreSQL® or MySQL® or a not only SQL (NoSQL) database such as MongoDB®.

Network 104 may be a communication network such as an enterprise intranet, a Wide-Area Network (WAN), a Local-Area Network (LAN), or another type of network. Network 104 may include a point-to-point connection, or another communications connection between two nodes. The network 104 may exist at a single geographical location, or may span multiple geographical locations.

Image acquisition device 106 may include imaging devices inside a medical organization or outside a medical organization (i.e. the imaging device may be remote, or located at a satellite clinic). While a single imaging acquisition device 106 is shown, it is understood that there may be a plurality of imaging devices.

Image acquisition device 106 may be located remotely from the medical organization or local to the medical organization. There may be one or more imaging device 106.

The one or more image acquisition devices 106 may be a variety of different imaging modalities that generate medical images such as X-ray Plain Film (PF) devices, digital X-ray devices, Computed Tomography (CT) devices, ultrasound devices, nuclear medicine imaging devices including Positron-Emission Tomography (PET) devices, Magnetic Resonance Imaging (MRI) devices, mammographic devices, or any other imaging modality used in a medical organization. The one or more image acquisition devices 106 may be mobile imaging devices such as mobile CT scanners. The medical images generated by the one or more imaging devices may be collected using analog means such as film and then subsequently scanned, or may initially be collected using digital sensor means such as a Charge Coupled Device (CCD). The one or more image acquisition devices 106 may operate to produce studies of patients of the medical organization. The one or more image acquisition devices 106 may collect various metadata at the time it captures images of the patient. The metadata collected by the one or more image acquisition devices 106 may be in DICOM format, HL7 format, or other formats for image data and associated metadata formats as are known. The metadata collected by the one or more image acquisition devices 106 may be entered at a user input device by a technician or clinician operating the image acquisition device. The one or more image acquisition devices 106 may include, for example, a General Electric® (GER) Revolution Apex® CT image acquisition device, a Siemens® Magnetom Vida® MR image acquisition device, and a Canon® UltiMax® x-ray image acquisition device.

Within a hospital organization, medical images (e.g., from CT or MRI scanners) are often stored in DICOM format in a PACS system such as enterprise imaging device 108. A medical image may be thought of as consisting of two parts: a metadata portion (also known as a metadata header) containing information about the image, for example, patient specific information, scanner setup, image sizes, etc., and the pixel data. The metadata may often be input by hand, and may be non-standard, varying from one scanner manufacturer to another and by institution. This means that it may be missing or erroneous.

The enterprise imaging device 108 may store medical image data collected at the one or more image acquisition devices 106, and image metadata corresponding to the medical image data.

The image data generated by the one or more image acquisition devices 106 and stored in the enterprise imaging device 108 may be provided in JPEG, lossless JPEG, Run-Length Encoding (RLE), JFIF, JPEG2000, Exif, GIF, BMP, PNG, PPM, PGM, PBM, PNM, WebP, HDR, HEIF, or any other known image format.

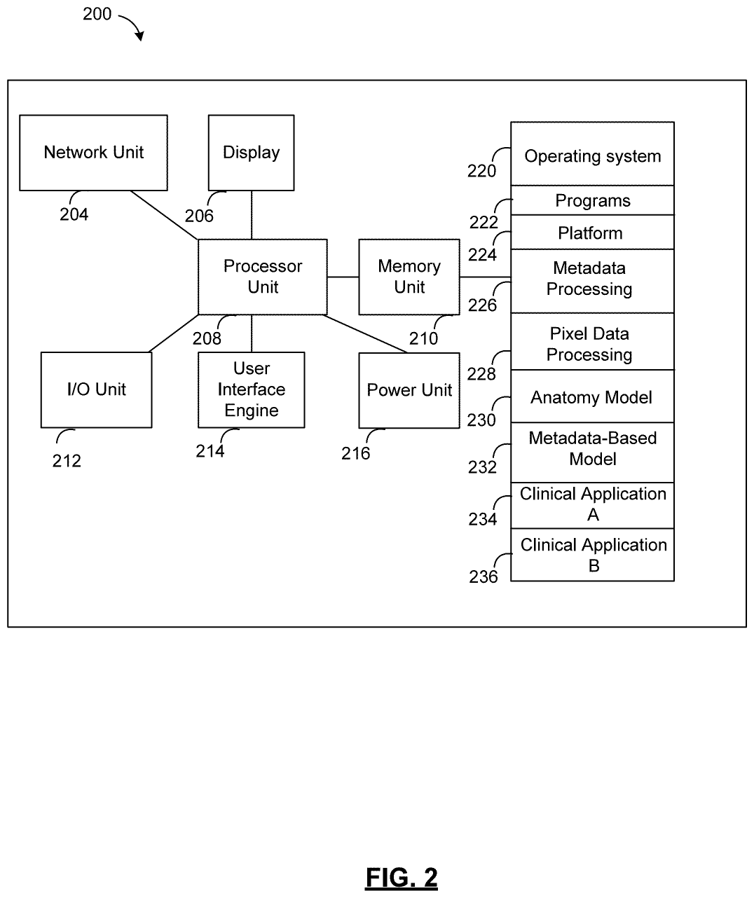

Reference is next made to FIG. 2, showing a block diagram 200 of the server 110 from FIG. 1. The processing server 200 has network unit 204, display 206, I/O unit 212, processor unit 208, memory unit 210, user interface engine 214, and power unit 216. The memory unit 210 has operating system 220, programs 222, anatomy engine 224, metadata processing 226, pixel data processing 228, anatomy model 230, metadata-based model 232, clinical application A 234 and clinical application B 236. The processing server 200 may be a virtual server on a shared host, or may itself be a physical server.

The network unit 204 may be a standard network adapter such as an Ethernet or 802.11x adapter. The processor unit 208 may include a standard processor, such as the Intel Xeon processor, for example. Alternatively, there may be a plurality of processors that are used by the processor unit 208 and may function in parallel. Alternatively there may be a plurality of processors including a Central Processing Unit (CPU) and a Graphics Processing Unit (GPU). The GPU may be, for example, from the GeForce® family of GPUs from Nvidia®, or the Radeon@ family of GPUs from AMD®. There may be a plurality of CPUs and a plurality of GPUs.

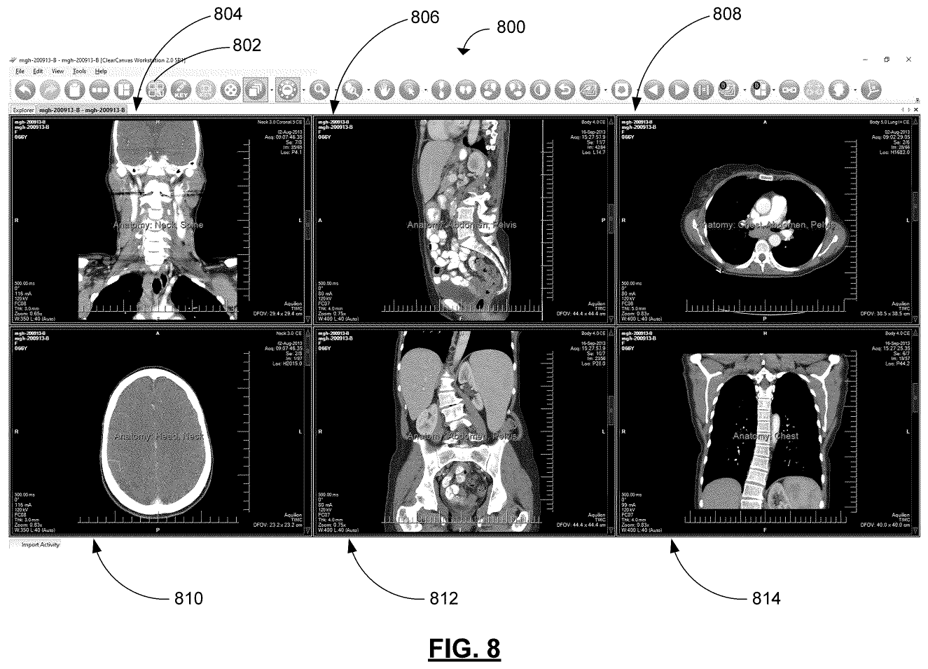

The processor unit 208 can also execute a user interface engine 214 that is used to generate various user interfaces, some examples of which are shown and described herein, such as in FIG. 8. The user interface engine 214 provides for clinical software application configuration layouts for users to configure clinical software applications, and clinician review user interfaces (e.g. a Hanging Protocol interface). User interface engine 214 may be an Application Programming Interface (API) or a Web-based application that is accessible via the network unit 204.

I/O unit 212 provides access to server devices including disks and peripherals. The I/O hardware provides local storage access to the programs running on processing server 200.

The power unit 216 provides power to the processing server 200.

Memory unit 210 may have an operating system 220, programs 222, anatomy engine 224, metadata processing 226, pixel data processing 228, anatomy model 230, metadata-based model 232, clinical application A 234 and clinical application B 236.

The operating system 220 may be a Microsoft Windows Server operating system, or a Linux-based operating system, or another operating system.

The programs 222 comprise program code that, when executed, configures the processor unit 208 to operate in a particular manner to implement various functions and tools for the server 200.

The platform 224 may be a software application for routing medical images and studies to the clinical application A 234 and the clinical application B 236. The platform 224 may also identify, query, receive, and assembled matching prior studies of the current study into a matching study set. The matching study set may be transmitted to the clinical software applications, and is provided to ensure a complete record of data is provided when the clinical applications perform their automated processing. In many medical organizations, the server 200 may require improved relevancy determinations of prior studies, or improved relevancy determinations for the clinical application to be used to process data. The improved relevancy may be determined for the data stored in the enterprise imaging system (see e.g. 108 in FIG. 1). The platform 224 may attempt to use the available metadata for a study or a medical image to identify the anatomy content. To do so, the platform 224 may require the anatomical view of the medical image or study. For reasons already mentioned above, often the metadata of a medical image or study is erroneous or missing. Further, in many circumstances, the platform 224 may only have access to a limited set of metadata, not all of the metadata fields available in the image header. For example, the platform might only have access to a few tags: Study Description, Series Description, Modality etc. Additionally, the available tags may differ from institution to institution and between different scanner (modality) manufacturers.

Metadata processing 226 may include preprocessing operations of the metadata. This can include object deserialization, data conversion, normalization, or other operations to prepare the metadata associated with images or studies for further processing by platform 224, anatomy model 230, metadata-based model 232, clinical application A 234 and clinical application B 236. Metadata pre-processing 226 may include determining the relevancy of study for later processing. Metadata pre-processing 226 may group images together-for example, a set of images may be grouped together if they belong to the same patient, belong in the same study, and/or belong to the same series, etc.

Metadata pre-processing 226 may determine some aspects of the type of the data in the current study. For example, if a series of images is of a scout/localizer type, if the type is a 2-d image, if it a primary image type or derived type.

Metadata pre-processing 226 may determine details about how the image acquisition device was configured when the data was captured (what were the settings etc.).

Metadata pre-processing 226 may determine the orientation of the data (was it captured top to bottom, left to right, back to front).

Metadata pre-processing 226 may determine whether the series of images form a ‘contiguous’ scan, and may determine if there gaps in the acquisition etc.

Metadata pre-processing 226 may determine whether or not the patient was injected with a contrast agent during scanning.

Metadata pre-processing 226 may determine the reason for the imaging (e.g. the tag ‘ProcedureStepDescription’ might describe the reason for the procedure-why was the patient imaged?).

Pixel data processing 228 may include preprocessing operations of the pixel data associated with the metadata. This can include formatting, cropping, data conversion, normalization, or other operations to prepare the pixel data for further processing by platform 224, anatomy model 230, clinical application A 234 and clinical application B 236.

Pixel data processing 228 may include decompression-often the pixel data is transmitted in a compressed format (e.g. jpg) and needs decompressed prior to use.

Pixel data processing 228 may include applying look-up-tables. Often the ‘raw’ pixel values may require rescaling and/or re-windowing to provide them in a ‘meaningful’ range.

Pixel data processing 228 may include reformatting-in some situations pixel data may be reformatted prior to using it in some applications (for example, the pixel data is often captured as a set of individual 2-d slices-one slice per instance), however, the data may be required in a 3-d block of pixels. To do this the pixels may be unpacked from each slice and ‘glued’ them together into a contiguous 3-d block of pixel data).

Pixel data processing 228 may include a spatial transformation-including pan, zoom, scale, rotate, flip etc. the pixel data prior to using it.

Pixel data processing 228 may include cropping-a region of the pixel data may be cropped as only a portion of the image may be used for subsequent processing.

Pixel data processing 228 may include creating a histogram-histograms of the pixel data values may be created to see the distribution of intensities prior to using the data.

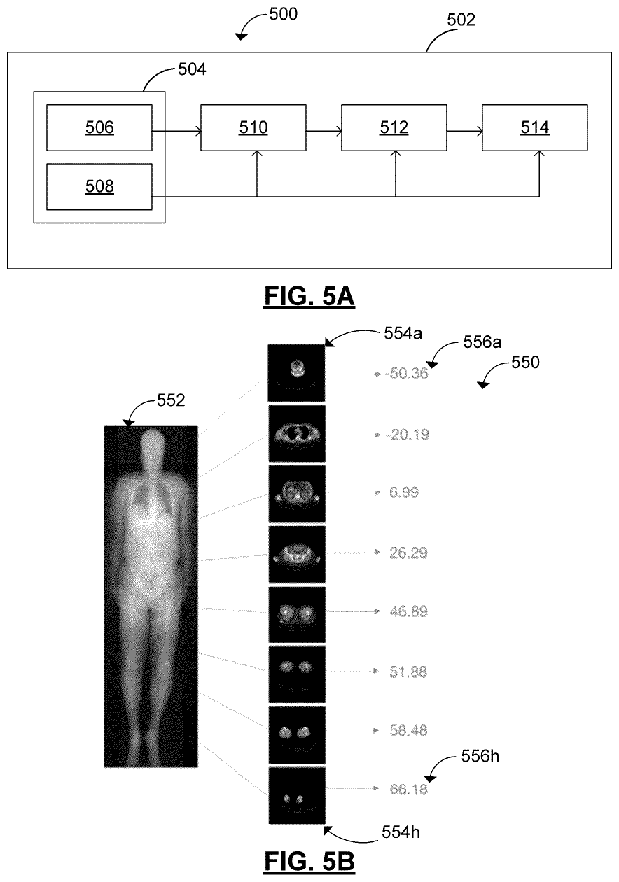

The anatomy model 230 may be a machine learning model or statistical model for identifying an anatomical view of a medical image based on the pixel data of the image. The anatomical location determined by the anatomy model may be determined based on the pixel data including a generated pixel-based anatomy prediction. The method takes single medical images, such as for example, CT slices from within the body and may output a number (see e.g. FIG. 5B) describing the predicted anatomical location of that medical image slice within the body.

The anatomy model 230 may be a Convolutional Neural Network (CNN), and may receive pixel data of medical images to output an anatomy score (see e.g. FIG. 5B). This score may be used to identify the anatomical location of the slice within the body.

The score output may be used to determine the location of the image in the body, and therefore, the anatomical content of the image (e.g. this slice contains the heart). Further, the score may be determined for a set of image slices and, based on all the ‘scores’ being similar, the score output may be used to determine that the set of slices includes the heart (i.e. use a set of scores to assign an anatomical location to a region of pixels).

The anatomy model 230 may be an Unsupervised Bodypart Regressor (UBR).

While UBR models including CNN and deep learning models may be used, the type of pixel-based model used may vary.

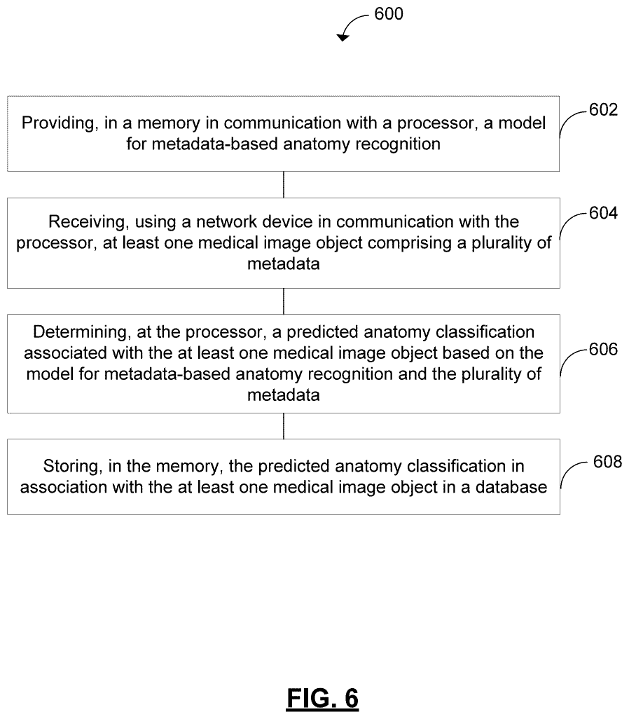

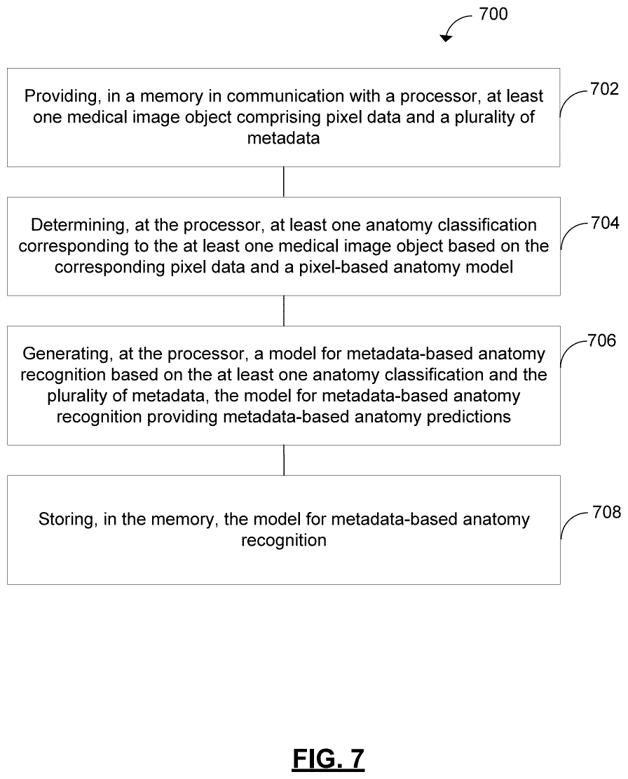

The metadata-based model 232 may be a machine learning model or statistical model for identifying an anatomical view of a medical image based on the image metadata of a medical image. The operation of the metadata-based model may be described in more detail in FIGS. 5A and FIG. 6. FIG. 7 describes how the metadata-based model may be generated.

Each of clinical software application A 234 and clinical software application B 236 is a software application that accepts an assembled matched study set and performs some processing. While only two clinical software applications are shown in FIG. 2, it is understood that there may be any number of clinical software applications, and potentially many more clinical software applications.

The clinical software applications may run in a process at processing server 200. In an alternate embodiment, the clinical software applications may run in a virtual machine (e.g. using VMware ESX) or a container (e.g. using Docker) at processing server 200. In another alternate embodiment, the clinical software applications may be located separately from processing server 200 in network communication with processing server 200, and in such a case the assembled matched study set may be transmitted to the clinical software application using network unit 204, and receive the processed study back from the clinical software application using network unit 204. In another alternate embodiment, a combination of clinical software application hosting may be used, for example a first clinical software application in one or more clinical software applications may run in a process at a processing server 200, a second clinical software application in the one or more clinical software applications may be located separately from the processing server 200, and a third clinical software application in the one or more clinical software applications may be located in a virtual machine or container at processing server 200.

In an alternate embodiment, the clinical software applications may be software modules provided via a remote cloud-hosted service which are accessed by using a remote API.

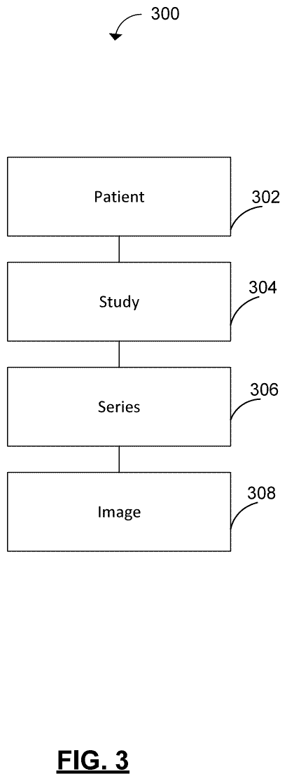

Referring next to FIG. 3, there is shown a block diagram 300 of a metadata object model. The metadata object model has patient entities 302, study entities 304, series entities 306, and image entities 308. The metadata object model may be normalized. The metadata object model may be used in association with a database at the processing server, a cache at the processing server, or another storage medium at the processing server to provide a relational database to store the incoming metadata.

Each of the patient entity 302, study entity 304, series entity 306 and image entity 308 may be composite entities that comprise other entities, other fields, or any other individual metadata fields as required.

A patient entity 302 may have a plurality of associated studies 304. A study may have a plurality of associated series 306. A series may have a plurality of associated images 308.

A patient entity 302 may have a plurality of metadata elements associated with it, including (but not limited to) the patient's name, a patient identifier, a patient's birth date, a patient's sex, and comments associated with the patient. The plurality of metadata elements in the patient entity 302 may have a user-entered source, a MWL source, or a combination thereof.

A study entity 304 may have a plurality of metadata elements associated with it, including (but not limited to) study instance identifier (or UID), a study date, a study time, a referring physician's name, a study identifier, an accession number, a study description, a referenced study sequence, a referenced SOP class identifier (or UID), and a reference SOP instance identifier (or UID). Other study metadata may include an admitting diagnosis description, a patient age, and a patient weight. The plurality of metadata elements in the study entity 304 may have a user-entered source, an automated source (for example, the date and time of a study may be automatically populated by an enterprise imaging device when the study is collected), a MWL source, or a combination thereof.

A series entity 306 may have a plurality of metadata elements associated with it, including (but not limited to) modality, series instance UID, series number, series date, series time, performing physician's name, protocol name, series description, operator's name, referenced performed procedure step sequence, referenced SOP class UID, referenced SOP instance UID, requested attributes sequence, requested procedure ID, scheduled procedure step ID, scheduled procedure step description, scheduled protocol code sequence, performed procedure step ID, performed procedure step start date, performed procedure step start time, performed procedure step description, performed protocol code sequence, and comments on the performed procedure step. The plurality of metadata elements in the series entity 306 may have a user-entered source, an automated source (for example, the date and time of a series may be automatically populated by an enterprise imaging device when the study is collected), a modality performed procedure step, a MWL source, or a combination thereof.

An image entity 308 may alternatively be referred to as a DICOM Service Object Pair (SOP) Instance. The SOP Instance is used to reference both image and non-image DICOM instances. The image entity 308 may have a plurality of metadata elements associated with it, including (but not limited to) manufacturer, institution name, station name, manufacturer's model name, device serial number, software version, private creator, equipment UID, and service UID. An image entity 308 may further have metadata including an application header sequence, an application header type, an application header ID, an application header version, workflow control flags, and archive management flags. There may further be modality (or image acquisition device) specific metadata in the image entity 308. The plurality of metadata elements in the image entity 308 may have an automated source (for example, the date and time of an image may be automatically populated by an enterprise imaging device when the study is collected), configuration based sources, or a combination thereof.

An entity may have a unique identifier. In the example of DICOM metadata, there may be a Unique Identifier, and the Unique Identifier may be globally unique across the entire DICOM environment.

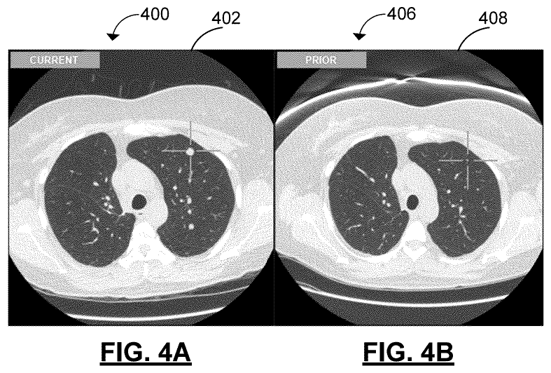

Referring to FIG. 4A, there is shown a diagram 400 of an example of medical image data 402 of a current study. The medical image data 402 of the current study may be an image collected by a CT image acquisition device (as shown) or an MRI image acquisition device. The image data may be collected in a variety of image formats as described herein.

Referring to FIG. 4B, there is shown a diagram 406 of an example of medical image data 408 of a prior study corresponding to the current study in FIG. 4A. The medical image data 408 of the current study may be an image collected by a CT image acquisition device (as shown) or an MRI image acquisition device. The image data may be collected in a variety of image formats as described herein.

Referring next to FIG. 5A and 5B together, there is shown a method diagram 500 and a pixel-based anatomy recognition diagram 550. The method 500 in FIG. 5A may be performed by the server 200 (see FIG. 2).

As described above, fetching pixel data to infer the anatomy of a study is expensive (in terms of elapsed time, processing effort and burden on existing systems such as the enterprise imaging system). Further, fetching pixel data just to infer anatomy may mean requesting and receiving significant amounts of pixel data that may not be required for processing.

Described herein are embodiments for generating and using a metadata-based anatomy model.

At 504, studies including medical images 506 and associated metadata 508 may be received. Where the metadata-based model is being generated, a plurality of medical studies or images may be received based on a query to an enterprise medical image device such as a PACS (see e.g. 108 in FIG. 1).

At 510, an anatomy score may be generated by the anatomy model based on pixel data 506 from a medical study or image 504.

To perform model training of the pixel-based model, the platform (see e.g. 224 in FIG. 2) may send a query to an enterprise imaging device (see e.g. 108 in FIG. 1) to request a plurality of medical images and accompanying metadata. The platform may initially request pixel data for studies frequently from the enterprise medical imaging device in order to reliably determine anatomy using an anatomy model (see e.g. 230 in FIG. 2) such as a UBR model. The request for pixel data may be scheduled for quiet times (e.g. during the night) in order reduce the load on the enterprise imaging system, e.g. PACS system, at busy times.

During model training and during prediction, the anatomy score may be generated for an image or study. The score may be a numerical score, for example, as shown in FIG. 5B, an anatomy score 556a may be generated based on a medical image 554a corresponding to a cross-sectional slice of the subject 552. Alternatively the anatomy score may be a categorical identifier, a text label, or another data type as known. Alternatively, the anatomy score may indicate a relative position along the body of the subject.

Optionally, a separate model or a rules-based scheme may be used to map the generated numerical score to an anatomy category.

Each score 556 may be associated with a medical image, and may correspond to a number that can be used to map the anatomical view of the image. The image 554a may generate score 556a of −50.36 which may correspond to a cross-sectional image of the subject's neck area. The image 556h may generate a score 554h of 66.18 which may correspond to a cross-sectional image of the subject's toes.

At 512, the score generated by the pixel-based anatomy model at 510 may be received by the metadata-based anatomy model (see e.g. 232 in FIG. 2). During model training, the metadata-based anatomy model receives the metadata associated with an image or study, along with the score generated by the pixel-based anatomy model at 510. The metadata-based model may ‘learn’ how to infer the anatomy of the study from the metadata, including DICOM tags. The metadata-based model may be individually trained for each medical organization, site, device, or location. Alternatively, trained metadata-based models for inferring relevancy or anatomy from metadata including DICOM tags at one site could be used to provide a starting point for training a model at another site (i.e., the delivery of models from one site to another may provide “transfer learning”) allowing the model to provide high quality predictions more quickly.

At 514, whilst inferring the anatomy using the pixel-based model, the metadata-based model may also perform a metadata-based prediction and compare the prediction results with the pixel-based prediction. This can include comparing the anatomical view predicted for the pixel-based model and the metadata-based model. Alternatively, this can include comparing the predicted clinical application (such as clinical application A 234 and clinical application B 236 in FIG. 2).

The comparison may be made using a rules-based scheme. For example, there may be a threshold identified between the prediction of the pixel-based model and the metadata-based model. Other rules may be used in order to identify situations where the pixel-based prediction or the metadata-based predictions is low, and thus the input and the respective model should be flagged for review.

In metadata-based predictions: there could be, for example, a first situation where 1 of many metadata tags has information that may enable determination of the body part (anatomical location) of the study. In an alternate situation, perhaps 5 or 10 tags may collectively indicate the same body part. In this alternate situation confidence in the metadata prediction of the anatomical content of the study may be higher than in the first situation having it indicated by a single tag. Similarly, if there are indications from multiple tags, but, some disagree with each other then the level of confidence in the metadata prediction decreases. In such a situation, a rule may be created to perform an election on the multiple tags to determine the comparison of the pixel-based and the metadata-based predictions. There may be an averaging conducted, if a confidence value is created. A threshold may be used in order to compare the prediction of one to the other.

In pixel-based prediction—due to the nature of the pixel-based model, it may generate a ‘confidence’ score which may indicate how confident it is about the pixel-based prediction of the body part in the study.

In one embodiment, if the metadata-based prediction is deemed low confidence (e.g. produced from 1 or a limited number of tags which may disagree) the rule may be defined to process the study using the pixel-based model. The output from both the metadata & pixel data predictions may be combined to create an overall prediction to improve the overall confidence in the prediction. In such a situation, the study may be flagged as an additional training case to improve the efficacy of the model based on future model training or generation.

In another embodiment, if the pixel-based prediction is deemed low confidence (e.g. less than some threshold) the study may be flagged so that the correct classification may be entered by a reviewer user, and so that the model may be improved in the future based on future model training or generation.

As the models are developed and improved over time, metadata-based predictions and pixel-based predictions may be continually produced, aiding the improvement of the metadata and pixel based predictions.

Subsequently, when a new study arrives, anatomy may be predicted based on the study metadata and the metadata-based model. Prior studies may be identified that have been reviewed having similar metadata. These previous studies may also be processed and compared with their pixel-based anatomy predictions, and a review may be conducted to determine if the metadata-based prediction and the pixel-based prediction match what is expected (e.g. the metadata predicted head, previous study data predicted head, so it is expected the pixel data determination to predict head, but it actually predicts neck). In such a case, a rule may be created that includes querying prior studies in addition to the current study as described, and if there is disagreement between the metadata-based prediction and the pixel-based prediction, then the respective current study or prior studies should be flagged for review. The studies flagged for review may be manually reviewed in order to determine the correct anatomy, and the models subsequently re-trained to improve future predictions.

The relevancy comparison between the pixel-based and the metadata-based prediction may be based upon other data, including for example, data from an auxiliary enterprise system such as a Modality Worklist (MWL) or a Radiology Information System (RIS).

To learn inferences from metadata, this may involve using a combination of more traditional data mining and machine learning techniques, for example, Recurrent Neural Networks (RNN) such as LSTM and other sequence models may be trained and used as the metadata-based model. The metadata model may also be a random forest model, a decision tree model, or a fully connected network model.

Over a time frame, the design described above may reduce the degree to which pixel data needs to be fetched to infer anatomy (since the metadata-based model may be able to do so accurately itself).

In one embodiment, feedback from the metadata-based prediction and the pixel-based prediction may be used to identify situations when the metadata-based model requires retraining. In an alternate embodiment, feedback from the metadata-based prediction and the pixel-based prediction may be used to identify situations when the pixel-based model requires retraining. For example, high confidence DICOM tag inferences may be used to flag (low confidence) UBR inferences that might indicate a need to update UBR training.

In this manner, the metadata-based model and the pixel-based model may cooperate to improve the quality and confidence of each other's predictions.

The metadata-based model may also be used, for example, to “clean up” metadata, including DICOM data, by replacing erroneous metadata including tags. It may further be used to add additional metadata to the medical images to aid other downstream systems (e.g. for hanging protocols).

Referring next to FIG. 6, there is shown a method diagram 600 for metadata-based anatomy recognition. The method 600 is for determining a predicted anatomy classification based on metadata, and may run on server 200 (see e.g. FIG. 2). The metadata may be associated with a study, and may be a DICOM format, an HL7 format, or another metadata format as known.

At 602, a model for metadata-based anatomy recognition is provided in a memory in communication with a processor. The model may be described as herein, and may be for different types of machine learning or statistical learning models.

Optionally, the model for metadata-based anatomy recognition may be a model for tag-based anatomy recognition, the plurality of metadata may be a plurality of tag-based metadata, and the predicted anatomy classification may be determined based on the model for tag-based anatomy recognition and the plurality of tag-based metadata.

Optionally, the model for tag-based anatomy recognition may include a Digital Imaging and Communications in Medicine (DICOM)-based model for tag-based anatomy recognition; the plurality of tag-based metadata may include a plurality of DICOM metadata; and the predicted anatomy classification may be determined based on the DICOM-based model for tag-based anatomy recognition and the plurality of DICOM metadata.

Optionally, the model for metadata-based anatomy recognition may include a Recurrent Neural Network (RNN) model and optionally the model may include a Long Short Term Memory (LSTM) model, a random forest model, a decision tree model, or a fully connected network model.

Optionally, the model for metadata-based anatomy recognition may be stored in a database on the server 200 (see e.g. FIG. 2).

Optionally, the model for metadata-based anatomy recognition may be stored on a filesystem.

Optionally, the model for metadata-based anatomy recognition may be received using a network device in communication with the processor.

At 604, at least one medical image object comprising a plurality of metadata is received using a network device in communication with the processor.

Optionally, the at least one medical image object may be received from a Picture Archiving and Communication Systems (PACS) server or a medical imaging device.

Optionally, the at least one medical image object may be received using a network device in communication with the processor.

At 606, a predicted anatomy classification associated with the at least one medical image object is determined, based on the model for metadata-based anatomy recognition and the plurality of metadata, at the processor.

Optionally, the predicted anatomy classification may be a numerical value associated with a position of an image slice (see e.g. FIG. 5B).

Optionally, the predicted anatomy classification may be a categorical value.

Optionally, the predicted anatomy classification may be a text value.

At 608, the predicted anatomy classification is stored in the memory in association with the at least one medical image object in a database.

Optionally, the predicted anatomy classification may be associated with the at least one medical image object in a database.

Optionally, the method may further include: generating a matched study set comprising the at least one medical image object and the predicted anatomy classification; determining a first clinical application based on the at least one medical image object and the model for metadata-based anatomy recognition; and transmitting the matched study set to the first clinical application. The matched study set may include other medical image objects (including pixel data and metadata) that may be identified based on a query to an enterprise imaging system based on the predicted anatomy and optionally further based on a subject identifier. In this way, the anatomy prediction may be used to identify other prior medical image objects such as studies that have been collected of the same anatomical view, or of the same subject. This may increase the number of matching studies in the matched study set, which is sent to the clinical application for processing. The selected clinical application may be identified or selected based on the prediction of the pixel-based model. The selected clinical application may be identified or selected based on the prediction of the metadata-based model.

Optionally, the method may further include: displaying, at a display device in communication with the processor, pixel data corresponding to the at least one medical image object; wherein the predicted anatomy classification may determine the display of the pixel data on the display device. For example, the predicted anatomy classification may determine the display of the pixel data on a “hanging protocol” (see e.g. the user interface in FIG. 8).

When hanging data, clinicians such as radiologists may prefer to display (or “hang”) both the ‘current’ study and any relevant prior studies. For example, if the current is a head CT and the patient has a previous head MR scan from 6 months previous they likely want to hang both these in the viewer for comparison.

The platform may identify the relevant prior studies for the current study. The relevancy may be anatomical (e.g. a chest MR is likely not a relevant prior for a head CT current). In this way, the relevancy of priors for display/hanging may depend on the determination of body part. So, if anatomy prediction is improved, the review by clinicians may consequently be improved because of the improved selection of relevant priors to display.

Optionally, the method may further include: determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network; comparing the predicted anatomy classification to the pixel-based predicted anatomy classification; and if the pixel-based predicted anatomy classification is different from the pixel-based predicted anatomy classification, the at least one medical image object may be flagged for review.

Optionally, the method may further include: determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network; comparing the predicted anatomy classification to the pixel-based predicted anatomy classification; and if the predicted anatomy classification is different from the pixel-based predicted anatomy classification, may automatically retrain the model for metadata-based anatomy recognition based on the pixel-based predicted anatomy classification and the at least one medical image object.

Optionally, the method may further include: determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network; comparing the predicted anatomy classification to the pixel-based predicted anatomy classification; and if the predicted anatomy classification is different from the pixel-based predicted anatomy classification, may automatically retrain the model for pixel-based anatomy recognition based on the predicted anatomy classification and the at least one medical image object.

Optionally, the method may further include: determining, at the processor, a pixel-based predicted anatomy classification and a second predicted clinical application from a model for pixel-based anatomy recognition wherein the model for pixel-based anatomy recognition optionally comprises an Unsupervised Body-part Regressor (UBR) or a Convolutional Neural Network; comparing the second predicted clinical application to the first predicted clinical application; and if the first predicted clinical application is different from the second predicted clinical application, the at least one medical image object may be flagged for review.

Optionally, the method may further include: determining, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network; comparing the second predicted clinical application to the first predicted clinical application; and if the first predicted clinical application is different from the second predicted clinical application, may automatically retrain the model for metadata-based anatomy recognition based on the pixel-based predicted anatomy classification and the at least one medical image object.

Optionally, the method may further include: determining, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network; comparing the second predicted clinical application to the first predicted clinical application; and if the first predicted clinical application is different from the second predicted clinical application, may automatically retrain the model for pixel-based anatomy recognition based on the predicted anatomy classification and the at least one medical image object.

Referring next to FIG. 7, there is shown a method diagram 700 for generating a model for metadata-based anatomy recognition.

At 702, at least one medical image object comprising pixel data and a plurality of metadata is provided in a memory in communication with a processor.

At 704, at least one anatomy classification corresponding to the at least one medical image object is determined at the processor, based on the corresponding pixel data and a pixel-based anatomy model.

Optionally, the pixel-based anatomy model may include an Unsupervised Body-part Regressor (UBR) or a Convolutional Neural Network.

At 706, a model for metadata-based anatomy recognition is generated at the processor based on the at least one anatomy classification and the plurality of metadata, the model for metadata-based anatomy recognition providing metadata-based anatomy predictions.

Optionally, the model for metadata-based anatomy recognition may include a Recurrent Neural Network (RNN) model and optionally a Long Short Term Memory (LSTM) model, a random forest model, a decision tree model, or a fully connected network model.

Optionally, the model for metadata-based anatomy recognition may include a model for tag-based anatomy recognition; the plurality of metadata may include a plurality of tag-based metadata; and the predicted anatomy classification may be determined based on the model for tag-based anatomy recognition and the plurality of tag-based metadata.

Optionally, the model for tag-based anatomy recognition may include a model for Digital Imaging and Communications in Medicine (DICOM)-based anatomy recognition; the plurality of tag-based metadata may include a plurality of DICOM metadata; and the predicted anatomy classification may be determined based on the model for DICOM-based anatomy recognition and the plurality of DICOM metadata.

At 708, the model for metadata-based anatomy recognition is stored in the memory.

Optionally, the method may further include: receiving the at least one medical image object from a PACS server or a medical imaging device using a network device in communication with the processor.

Referring next to FIG. 8, there is shown a user interface diagram 800 in accordance with one or more embodiments. The user interface 800 may be for clinician review of medical images, e.g. a “hanging protocol” as generally known.

The display of medical images for a subject may be predetermined in several boxes in a grid. For example, a hanging protocol may show a top row having a top-left image 804 of a “neck spine” anatomical view, a top-middle image 806 of an “abdomen pelvis” anatomical view, a top-right image 808 of a “chest abdomen” anatomical view, a bottom-left image 810 of a “head neck” anatomical view, a bottom-middle image 812 of an “abdomen pelvis” anatomical view, and a bottom right image 814 of a “chest” anatomical view. The anatomical views used to assign a viewing position (e.g. “top-left”, “top-middle”, “top-right”, “bottom-left”, “bottom-middle”, and “bottom-right”) for each image in the user interface may be identified automatically using anatomy predictions of the metadata-based anatomy model described herein. The anatomical views used to assign a viewing position (e.g. “top-left” box, “top-middle” box, “top-right” box, “bottom-left” box, “bottom-middle” box, and “bottom-right” box) for each image in the user interface may be identified automatically using a combination of the anatomical prediction of the metadata-based anatomy model and the pixel-based anatomy model described herein.

A toolbar 802 may allow for various features and functions associated with the user interface. For example, reviewing a worklist, zooming in on images, annotating images, etc.

A user of the user interface (the “hanging protocol”) may create one or more user-configurable “hanging protocols” by assigning configurable rules to a fixed set of views. The user may assign a rule to each of the boxes (views) on the user interface (e.g. When encountering a particular study, the user prefers the top left view/box to contain the chest CT image series). It may then the responsibility of the underlying viewer software to take the anatomical prediction of the images based on the pixel-based prediction and/or the metadata-based prediction for display. Where the pixel-based prediction and/or the metadata-based prediction indicate a set of images to be ‘chest CT’, then the rule may assign that image to the top left view/box.

The fixed set of images may be displayed using the configurable rules such that particular anatomical views may consistently appear in the same location in the grid of the images. In this manner, a user may efficiently select how they desire to review medical images, and save time in the review of images associated by a subject by reducing the amount of repositioning time required to get images without anatomical view predictions in order to prepare for a particular subject's review.

When hanging data, clinicians such as radiologists may prefer to display (or “hang”) both the ‘current’ study and any relevant prior studies. For example, if the current is a head CT and the patient has a previous head MR scan from 6 months previous they likely want to hang both these in the viewer for comparison.

The platform may identify the relevant prior studies for the current study. The relevancy may be anatomical (e.g. a chest MR is likely not a relevant prior for a head CT current). In this way, the relevancy of priors for display/hanging may depend on the determination of body part. So, if anatomy prediction is improved, the review by clinicians may consequently be improved because of the improved selection of relevant priors to display.

The present invention has been described here by way of example and with reference to several example embodiments. These embodiments are merely exemplary and do not limit the scope of the invention, which is limited only by claims.

We claim:

1. A computer-implemented method for metadata-based anatomy recognition, comprising:
providing, in a memory in communication with a processor, a model for metadata-based anatomy recognition;
receiving, using a network device in communication with the processor, at least one medical image object comprising a plurality of metadata;
determining, at the processor, a predicted anatomy classification associated with the at least one medical image object based on the model for metadata-based anatomy recognition and the plurality of metadata; and
storing, in the memory, the predicted anatomy classification in association with the at least one medical image object in a database.

2. The method of claim 1, wherein:
the model for metadata-based anatomy recognition comprises a model for tag-based anatomy recognition;
the plurality of metadata comprises a plurality of tag-based metadata; and
the predicted anatomy classification is determined based on the model for tag-based anatomy recognition and the plurality of tag-based metadata.

3. The method of claim 2, wherein:
the model for tag-based anatomy recognition comprises a Digital Imaging and Communications in Medicine (DICOM)-based model for tag-based anatomy recognition;
the plurality of tag-based metadata comprises a plurality of DICOM metadata; and
the predicted anatomy classification is determined based on the DICOM-based model for tag-based anatomy recognition and the plurality of DICOM metadata.

4. The method of claim 1, further comprising:
generating a matched study set comprising the at least one medical image object and the predicted anatomy classification;
determining a first clinical application based on the at least one medical image object and the model for metadata-based anatomy recognition; and
transmitting the matched study set to the first clinical application.

5. The method of claim 1, further comprising:
displaying, at a display device in communication with the processor, pixel data corresponding to the at least one medical image object;
wherein the predicted anatomy classification determines the display of the pixel data on the display device.

6. The method of claim 1, wherein the at least one medical image object is received from a Picture Archiving and Communication Systems (PACS) server or a medical imaging device.

7. The method of claim 1, further comprising:
determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network;
comparing the predicted anatomy classification to the pixel-based predicted anatomy classification; and
if the pixel-based predicted anatomy classification is different from the pixel-based predicted anatomy classification, flagging the at least one medical image object for review.

8. The method of claim 1, further comprising:
determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network;
comparing the predicted anatomy classification to the pixel-based predicted anatomy classification; and
if the predicted anatomy classification is different from the pixel-based predicted anatomy classification, automatically retraining the model for metadata-based anatomy recognition based on the pixel-based predicted anatomy classification and the at least one medical image object.

9. The method of claim 1, further comprising:
determining, at the processor, a pixel-based predicted anatomy classification from a model for pixel-based anatomy recognition, wherein the model for pixel-based anatomy recognition optionally comprises an Unsupervised Body-part Regressor (UBR) or a Convolutional Neural Network;
comparing the predicted anatomy classification to the pixel-based predicted anatomy classification; and
if the predicted anatomy classification is different from the pixel-based predicted anatomy classification, automatically retraining the model for pixel-based anatomy recognition based on the predicted anatomy classification and the at least one medical image object.

10. The method of claim 4, further comprising:
determining, at the processor, a pixel-based predicted anatomy classification and a second predicted clinical application from a model for pixel-based anatomy recognition wherein the model for pixel-based anatomy recognition optionally comprises an Unsupervised Body-part Regressor (UBR) or a Convolutional Neural Network;
comparing the second predicted clinical application to the first predicted clinical application; and
if the first predicted clinical application is different from the second predicted clinical application, flagging the at least one medical image object for review.

11. The method of claim 4, further comprising:
determining, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network;
comparing the second predicted clinical application to the first predicted clinical application; and
if the first predicted clinical application is different from the second predicted clinical application, automatically retraining the model for metadata-based anatomy recognition based on the pixel-based predicted anatomy classification and the at least one medical image object.

12. The method of claim 4, further comprising:
determining, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network;
comparing the second predicted clinical application to the first predicted clinical application; and
if the first predicted clinical application is different from the second predicted clinical application, automatically retraining the model for pixel-based anatomy recognition based on the predicted anatomy classification and the at least one medical image object.

13. The method of claim 1, wherein the model for metadata-based anatomy recognition comprises a Recurrent Neural Network (RNN) model, and optionally a Long Short Term Memory (LSTM) model, a random forest model, a decision tree model, or a fully connected network model.

14. A computer-implemented system for metadata-based anatomy recognition, comprising:
a memory, comprising:
a model for metadata-based anatomy recognition;
a network device, and
a processor, the processor configured to:
receive, from the network device, at least one medical image object comprising a plurality of metadata;
determine a predicted anatomy classification associated with the at least one medical image object based on the model for metadata-based anatomy recognition and the plurality of metadata; and
store, in the memory, the predicted anatomy classification in association with the at least one medical image object in a database.

15. The system of claim 14, wherein:
the model for metadata-based anatomy recognition comprises a model for tag-based anatomy recognition;
the plurality of metadata comprises a plurality of tag-based metadata; and
the predicted anatomy classification is determined based on the model for tag-based anatomy recognition and the plurality of tag-based metadata.

16. The system of claim 15, wherein:
the model for tag-based anatomy recognition comprises a Digital Imaging and Communications in Medicine (DICOM)-based model for tag-based anatomy recognition;
the plurality of tag-based metadata comprises a plurality of DICOM metadata; and
the predicted anatomy classification is determined based on the DICOM-based model for tag-based anatomy recognition and the plurality of DICOM metadata.

17. The system of claim 14, wherein the processor is further configured to:
generate a matched study set comprising the at least one medical image object and the predicted anatomy classification;
determine a first clinical application based on the at least one medical image object and the model for metadata-based anatomy recognition; and
transmit the matched study set to the first clinical application.

18. The system of claim 14, further comprising:
a display device in communication with the processor;
wherein the processor is further configured to:
display, at the display device, pixel data corresponding to the at least one medical image object; and
wherein the predicted anatomy classification determines the display of the pixel data on the display device.

19. The system of claim 14, wherein the at least one medical image object is received from a Picture Archiving and Communication Systems (PACS) server or a medical imaging device.

20. The system of claim 14, wherein the processor is further configured to:
determine a pixel-based predicted anatomy classification from a model for pixel-based anatomy recognition, wherein the model for pixel-based anatomy recognition optionally comprises an Unsupervised Body-part Regressor (UBR) or a Convolutional Neural Network;
compare the predicted anatomy classification to the pixel-based predicted anatomy classification; and
if the pixel-based predicted anatomy classification is different from the pixel-based predicted anatomy classification, flagging the at least one medical image object for review.

21. The system of claim 14, wherein the processor is further configured to:
determine a pixel-based predicted anatomy classification from a model for pixel-based anatomy recognition, wherein the model for pixel-based anatomy recognition optionally comprises an Unsupervised Body-part Regressor (UBR) or a Convolutional Neural Network;
compare the predicted anatomy classification to the pixel-based predicted anatomy classification; and
if the predicted anatomy classification is different from the pixel-based predicted anatomy classification, automatically retraining the model for metadata-based anatomy recognition based on the pixel-based predicted anatomy classification and the at least one medical image object.

22. The system of claim 14, wherein the processor is further configured to:
determine a pixel-based predicted anatomy classification from a model for pixel-based anatomy recognition, wherein the model for pixel-based anatomy recognition optionally comprises an Unsupervised Body-part Regressor (UBR) or a Convolutional Neural Network;
compare the predicted anatomy classification to the pixel-based predicted anatomy classification; and
if the predicted anatomy classification is different from the pixel-based predicted anatomy classification, automatically retraining the model for pixel-based anatomy recognition based on the predicted anatomy classification and the at least one medical image object.

23. The system of claim 17, wherein the processor is further configured to:
determine a pixel-based predicted anatomy classification and a second predicted clinical application from a model for pixel-based anatomy recognition wherein the model for pixel-based anatomy recognition optionally comprises an Unsupervised Body-part Regressor (UBR) or a Convolutional Neural Network;
compare the second predicted clinical application to the first predicted clinical application; and
if the first predicted clinical application is different from the second predicted clinical application, flagging the at least one medical image object for review.

24. The system of claim 17, wherein the processor is further configured to:
determine a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network;
compare the second predicted clinical application to the first predicted clinical application; and
if the first predicted clinical application is different from the second predicted clinical application, automatically retraining the model for metadata-based anatomy recognition based on the pixel-based predicted anatomy classification and the at least one medical image object.

25. The system of claim 17, wherein the processor is further configured to:
determine, at the processor, a pixel-based predicted anatomy classification and a second predicted clinical application from a model for pixel-based anatomy recognition, wherein the model for pixel-based anatomy recognition optionally comprises an Unsupervised Body-part Regressor (UBR) or a Convolutional Neural Network;
compare the second predicted clinical application to the first predicted clinical application; and
if the first predicted clinical application is different from the second predicted clinical application, automatically retrain the model for pixel-based anatomy recognition based on the predicted anatomy classification and the at least one medical image object.

26. The system of claim 14, wherein the model for metadata-based anatomy recognition comprises a Recurrent Neural Network (RNN) model, and optionally a Long Short Term Memory (LSTM) model, a random forest model, a decision tree model, or a fully connected network model.

27. A computer-implemented method for generating a model for metadata-based anatomy recognition, comprising:
providing, in a memory in communication with a processor, at least one medical image object comprising pixel data and a plurality of metadata;
determining, at the processor, at least one anatomy classification corresponding to the at least one medical image object based on the corresponding pixel data and a pixel-based anatomy model;
generating, at the processor, a model for metadata-based anatomy recognition based on the at least one anatomy classification and the plurality of metadata, the model for metadata-based anatomy recognition providing metadata-based anatomy predictions; and
storing, in the memory, the model for metadata-based anatomy recognition.

28. The method of claim 27, further comprising:
receiving the at least one medical image object from a PACS server or a medical imaging device using a network device in communication with the processor.

29. The method of claim 27, wherein:
the model for metadata-based anatomy recognition comprises a model for tag-based anatomy recognition;
the plurality of metadata comprises a plurality of tag-based metadata; and
the predicted anatomy classification is determined based on the model for tag-based anatomy recognition and the plurality of tag-based metadata.

30. The method of claim 29, wherein:
the model for tag-based anatomy recognition comprises a model for Digital Imaging and Communications in Medicine (DICOM)-based anatomy recognition;
the plurality of tag-based metadata comprises a plurality of DICOM metadata; and
the predicted anatomy classification is determined based on the model for DICOM-based anatomy recognition and the plurality of DICOM metadata.

31. The method of claim 27, wherein the pixel-based anatomy model comprises an Unsupervised Body-part Regressor (UBR) or a Convolutional Neural Network.

32. The method of claim 27, wherein the model for metadata-based anatomy recognition comprises a Recurrent Neural Network (RNN) model, and optionally a Long Short Term Memory (LSTM) model, a random forest model, a decision tree model, or a fully connected network model.

33. A computer-implemented system for generating a model for metadata-based anatomy recognition, comprising:
a memory comprising:
at least one medical image object comprising:
pixel data, and
a plurality of metadata;
a network device, and
a processor configured to:
determine at least one anatomy classification corresponding to the at least one medical image object based on the corresponding pixel data and a pixel-based anatomy model;
generate a model for metadata-based anatomy recognition based on the at least one anatomy classification and the plurality of metadata, the model for metadata-based anatomy recognition providing metadata-based anatomy predictions; and
store, in the memory, the model for metadata-based anatomy recognition.

34. The system of claim 33, wherein the processor is further configured to:
receive the at least one medical image object from a PACS server or a medical imaging device using the network device.

35. The system of claim 33, wherein:
the model for metadata-based anatomy recognition comprises a model for tag-based anatomy recognition;
the plurality of metadata comprises a plurality of tag-based metadata; and
the predicted anatomy classification is determined based on the model for tag-based anatomy recognition and the plurality of tag-based metadata.

36. The system of claim 35, wherein:
the model for tag-based anatomy recognition comprises a model for Digital Imaging and Communications in Medicine (DICOM)-based anatomy recognition;
the plurality of tag-based metadata comprises a plurality of DICOM metadata; and
the predicted anatomy classification is determined based on the model for DICOM-based anatomy recognition and the plurality of DICOM metadata.

37. The system of claim 33, wherein the pixel-based anatomy model comprises an Unsupervised Body-part Regressor (UBR) or a Convolutional Neural Network.

38. The system of claim 33, wherein the model for metadata-based anatomy recognition comprises a Recurrent Neural Network (RNN) model, and optionally a Long Short Term Memory (LSTM) model, a random forest model, a decision tree model, or a fully connected network model.
